(19) United States
(12) Patent Application Publication
(10) Pub. No.: US 2011/0030680 A1
(43) Pub. Date: Feb. 10, 2011

(54) TRACHEAL TUBE WITH DRUG DELIVERY DEVICE AND METHOD OF USING THE SAME

(75) Inventors: Lockett E. Wood, Lyons, CO (US); Sarah Spence, Golden, CO (US)

Correspondence Address: NELLCOR PURITAN BENNETT LLC;ATTN: IP LEGAL, 6135 Gunbarrel Avenue, Boulder, CO, 80301, US

(73) Assignee: Nellcor Puritan Bennett LLC, Boulder, CO (US)

(21) Appl. No.: 12/846,581

(22) Filed: Jul. 29, 2010

Related U.S. Application Data
(60) Provisional application No. 61/229,974, filed on Jul. 30, 2009.

(51) Int. Cl. A61M 16/04; A61M 11/00

(52) U.S. Cl. 128/200.23; 128/203.12

(57) ABSTRACT

According to various embodiments, a tracheal tube may include a drug delivery system for nebulizing drug to droplets within a particular drop size range. The drug delivery system may include an integral nebulizer located towards a distal end of the tracheal tube. The nebulizer may comprise a micropump and may be in fluid communication with a drug delivery lumen, which in turn may be coupled to a system for monitoring the delivery of drug to the nebulizer. The nebulizer may be sized and shaped to be accommodated within a lumen that runs alongside a main respiratory lumen of the tracheal tube.

### CROSS-REFERENCE TO RELATED APPLICATIONS

This application claims priority to U.S. Provisional Application No. 61/229,974, filed Jul. 30, 2009, the disclosure of which is hereby incorporated by reference in its entirety for all purposes.

### BACKGROUND

The present disclosure relates generally to medical devices and, more particularly, to airway devices, such as tracheal tubes.

This section is intended to introduce the reader to aspects of the art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the course of treating a patient, a tube or other medical device may be used to control the flow of air, food, fluids, or other substances into the patient. For example, tracheal tubes may be used to control the flow of air or other gases through a patient's trachea. Such tracheal tubes may include endotracheal (ET) tubes, tracheotomy tubes, or transtracheal tubes. In many instances, it is desirable to provide a seal between the outside of the tube or device and the interior of the passage in which the tube or device is inserted. In this way, substances can only flow through the passage via the tube or other medical device, allowing a medical practitioner to maintain control over the type and amount of substances flowing into and out of the patient.

Physicians may wish to deliver drugs to a ventilated patient, in particular because critically ill patients are often at risk of developing secondary infections. For example, drugs may be delivered directly through the airway circuit of a ventilated patient. In particular, a nebulizer may be placed in-line with the inspiratory limb of the ventilation circuit. The respiratory gases mix with the nebulized drug in the upstream portion of the ventilation circuit and, when the respiratory gases are taken into the lungs, the drug is also delivered to the airway. However, nebulizers may interfere with efficient delivery of respiratory gases because the nebulizers entrain air with the nebulization, which in turn may interfere with ventilator settings.

### BRIEF DESCRIPTION OF THE DRAWINGS

With reference to the drawings in which:

FIG. 1 illustrates an exemplary system including a tracheal tube with a drug delivery system;

FIG. 2 is an elevation view of an exemplary tracheal tube that may be used in conjunction with the system of FIG. 1;

FIG. 3 is a detail view of the distal end of the tracheal tube of FIG. 2;

FIG. 4 is a cross-sectional view along an axis of a drug delivery lumen of an exemplary drug delivery system located at the distal end of a tracheal tube; and

FIG. 5 is an elevation view of an exemplary drug delivery control system that may be used in conjunction with the tracheal tube of FIG. 2.

### DETAILED DESCRIPTION OF SPECIFIC EMBODIMENTS

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

A tracheal tube may be used to seal a patient's airway and provide positive pressure to the lungs. During ventilation, a physician may wish to provide drug delivery to the lungs along with respiratory gases. In this manner, a drug may be targeted directly to the patient's airway tissue, which may be vulnerable to infection during ventilation. Often, a nebulizer may be coupled to the airway circuit at a point upstream of the tracheal tube. The nebulizer converts a drug supply to a mist or spray, which may be mixed with respiratory gases and pushed into the lungs through the tracheal tube. However, these nebulizers often interfere with ventilator settings because they entrain air during the nebulization process, which may result in inaccurate calculations of pressures within the respiratory circuit as well as inaccurate calculations of the concentrations of particular respiratory gases. In addition, these techniques compromise effective medication delivery and waste medication. For example, since nebulization in the inspiratory limb is estimated to have efficiencies of between 2% to 30%, much of the medication is lost within the respiratory circuit.

Provided herein are tracheal tubes that include nebulizers located at the distal end of the tracheal tubes. By nebulizing drugs, which may for example comprise or be dispersed in fluid medium, closer to the point of drug delivery, less drug is lost to the system and wasted. Further, because the nebulizing takes place outside the respiratory circuit, and, in particular embodiments, may not involve entraining a significant amount of air, the percentage of air entrained into the respiratory circuit is reduced. In addition, such nebulizers may provide tighter control of drop size of the nebulized spray. Because the drop size is related to a desired drug delivery site, a nebulizer with good drop size control allows more specific drug targeting. For example, smaller droplets tend to follow the inhaled airflow and reach deeper and narrower regions of the respiratory tract. Furthermore, for certain medical conditions it is desirable for a drug to be deposited in the bronchi, rather than the alveoli. For applications of this type, a somewhat larger droplet size, for example, may be beneficial. Accordingly, the deposition site of the nebulized spray depends on the geometry of the respiratory tract and the droplet size or droplet spectrum of the spray.

In embodiments described herein, the disclosed tracheal tubes, systems, and methods may be used in conjunction with any appropriate medical device, including without limitation a feeding tube, an endotracheal tube, a Broncho-Cath™ tube, a tracheotomy tube, a circuit, an airway accessory, a connector, an adapter, a filter, a humidifier, a nebulizer, nasal cannula, or a supraglottic mask/tube. The present techniques may also be used to monitor any patient benefiting from mechanical ventilation, e.g., positive pressure ventilation. Further, the devices and techniques provided herein may be used to monitor a human patient, such as a trauma victim, an intubated patient, a patient with a tracheotomy, an anesthetized patient, a cardiac arrest victim, a patient suffering from airway obstruction, or a patient suffering from respiratory failure.

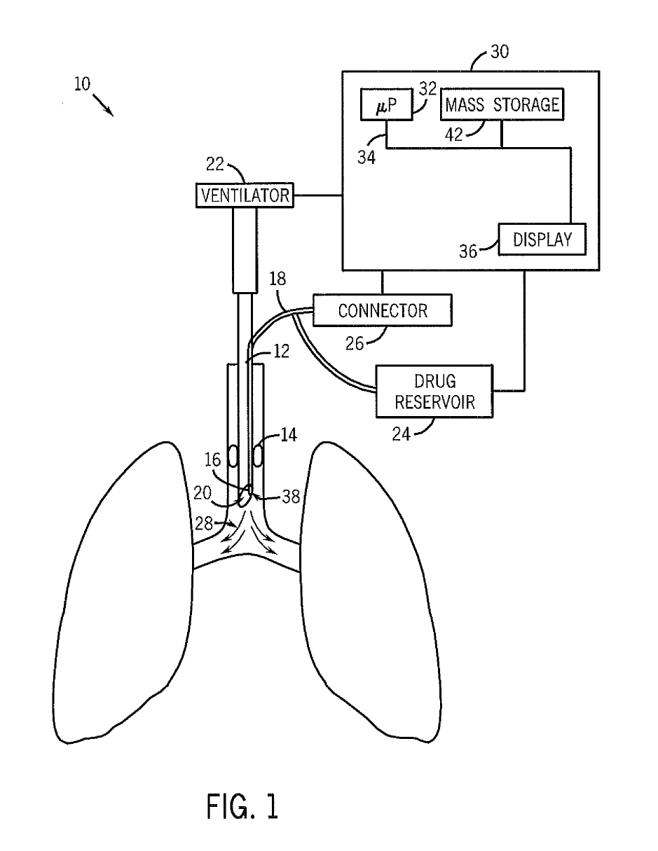

FIG. 1 shows an exemplary tracheal tube system 10 that has been inserted into a patient's trachea. The system 10 includes a tracheal tube 12, shown here as endotracheal tube, with an inflatable balloon cuff 14 that is disposed on walls of the tube 12 that may be inflated to form a seal against tracheal walls. The tracheal tube 12 is configured to deliver respiratory gases into the lungs and to allow the expiration of gases from the lungs. When mechanical ventilation is provided via the tube system, a ventilator 22 is typically provided, such as those available from Nellcor Puritan Bennett LLC. The ventilator 22 may be connected to the tube 12 through any appropriate configuration of connectors and/or tubing for delivering respiratory gases into the lungs.

As shown, the tracheal tube 12 may also include a nebulizer 16 for delivering drugs to the lungs. It should be understood that a drug may, in particular embodiments, be any substance that a clinician may wish to deliver to a patient, including any substance that is intended for use in the diagnosis, cure, mitigation, treatment, or prevention of a clinical condition, or may be any substance that affects the structure or function of the body. Further, the drug may be provided to the nebulizer 16 in any suitable formulation, including dispersed within a liquid. The nebulizer 16 may be formed in or disposed within a drug delivery lumen 18 towards the distal end 20 of the tracheal tube 12. The drug delivery lumen 18 may be coupled to a drug reservoir 24 by any appropriate connectors and/or tubing. The drug reservoir 24 may be configured to hold an appropriate volume of a drug for converting to a spray by the nebulizer 16. The drug may be transferred from the drug reservoir to the distal end 38 of to drug delivery lumen 18, either by gravity or through a motive force. In alternative embodiments, the drug delivery lumen 18 may be coupled to an injection port and the drug may be injected down into the drug delivery lumen 18 via a removable drug reservoir 24, such as an injection syringe, that also provides a motive force.

In addition, the nebulizer 16 may be coupled by appropriate leads or wires to a connector 26, which in turn may be coupled to an appropriate port on a patient monitor 30. The monitor 30 is configured to implement embodiments of the present disclosure to control the nebulizer 16. It should be understood that the monitor 30 may be a stand-alone device or may, in embodiments, be integrated into a single device with, for example, the ventilator 22. The monitor 32 may include processing circuitry, such as a microprocessor 32 coupled to an internal bus 34 and a display 36. In an embodiment, the monitor 30 may be configured to communicate with the nebulizer 16, for example via connector 26, to activate the nebulizer 16 at the appropriate times, e.g., when there is fluid in the drug reservoir 24 or only during certain portions of the ventilation cycle. In certain embodiments, the connector 26 may also provide calibration information for the tube 12 and/or the nebulizer 16. The information may then be stored in mass storage device 42, such as RAM, PROM, optical storage devices, flash memory devices, hardware storage devices, magnetic storage devices, or any suitable computer-readable storage medium. The information may be accessed and operated upon according to microprocessor 32 instructions. The monitor 32 may be configured to provide indications related to operation of the nebulizer 16, including an estimate of the delivery rate of the fluid, such as an audio, visual or other indication that the nebulizer 16 is providing drug, or may be configured to communicate the information to another device, such as the ventilator 22.

The connector 26 may be suitably configured to connect to a receiving port on the monitor 30. The connector 26 may contain an information element, such as a memory circuit, e.g., an EPROM, EEPROM, coded resistor, or flash memory device for storing calibration information. The connector 26 may also contain certain processing circuitry for providing a drive signal to the nebulizer 16. When the connector 26 is coupled to the monitor 30, the information element may be accessed to provide calibration information to the monitor 30. In certain embodiments, calibration information may be provided in a barcode on the tube or associated packaging that may be scanned by a reader coupled to the monitor 30.

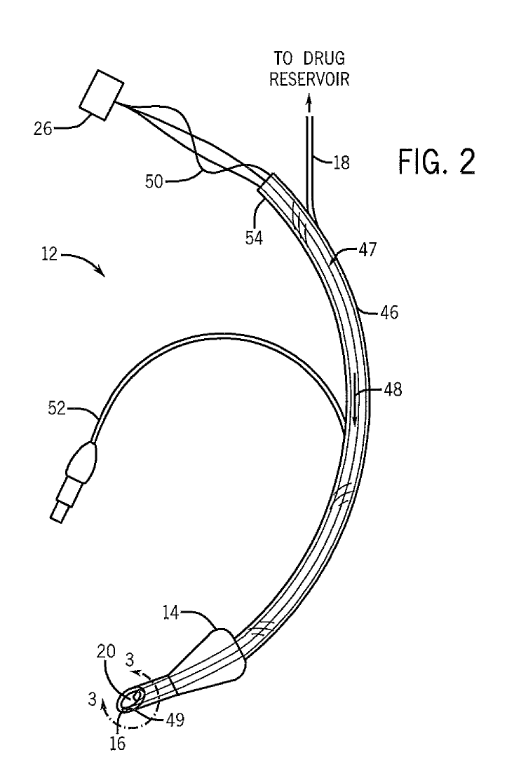

FIG. 2 is an elevation view of an exemplary tracheal tube 12 according to certain embodiments. As noted, the tube 12 may include a drug delivery lumen 18 disposed on or in a wall 46 of the tube. The drug delivery lumen 18 may run substantially parallel to the main respiratory lumen 47 of the tube 12. The tube walls 46 define an airway flow path for delivering respiratory fluids (e.g., gases, liquids, etc.) to a patient's lungs (as shown by arrow 48) and for allowing gases to flow out of the lungs. The drug delivery lumen 18 may be formed in the wall of the tube and may terminate in an opening 49. The nebulizer 16 is disposed within the drug delivery lumen 18, towards its distal end 38. The nebulizer 16 may be adhered or otherwise applied to the lumen walls, as discussed below. The tube 12 also includes an inflatable cuff 14, which may be inflated via a separate inflation lumen 52.

In addition, the tube 12 may include connector 26, which may be coupled to the tube 12 by the appropriate wires or leads 50. For example, in particular embodiments, the nebulizer 16 may be coupled to two wires 50. In other embodiments, the nebulizer 16 may be arranged in a three-wire or four-wire configuration, depending on, for example, the particular arrangement of the pumping elements. The wires 50 may be disposed within the drug delivery lumen 18 (e.g., may be threaded into the lumen 18) or, in other embodiments, may be embedded within the walls 46 of the tube 12. In such an arrangement, the wires 50 may run alongside the lumen 18, and may emerge from the tube 12 towards its proximal end 54. Such an arrangement, in which the wires 50 are at least partially embedded in the walls 46 of the tube 12, may serve to anchor the nebulizer 16 in place. The wires 50 may be coupled to the connector 26 by any suitable arrangement.

The tube 12 and the cuff 14 may be formed from materials having suitable mechanical properties (such as puncture resistance, pin hole resistance, tensile strength), chemical properties (such as biocompatibility). In one embodiment, the walls of the cuff 14 are made of a polyurethane having suitable mechanical and chemical properties. An example of a suitable polyurethane is Dow Pellethane® 2363-80A. In another embodiment, the walls of the cuff 14 are made of a suitable polyvinyl chloride (PVC). In one embodiment, the cuff 14 may be generally sized and shaped as a high volume, low pressure cuff that may be designed to be inflated to pressures between about 15 cm H₂O and 30 cm H₂O. The system 10 may also include a respiratory circuit connected to the endotracheal tube 12 that allows one-way flow of expired gases away from the patient and one-way flow of inspired gases towards the patient. The respiratory circuit, including the tube 12, may include standard medical tubing made from suitable materials such as polyurethane, polyvinyl chloride (PVC), polyethylene teraphthalate (PETP), low-density polyethylene (LDPE), polypropylene, silicone, neoprene, polytetrafluoroethylene (PTFE), or polyisoprene.

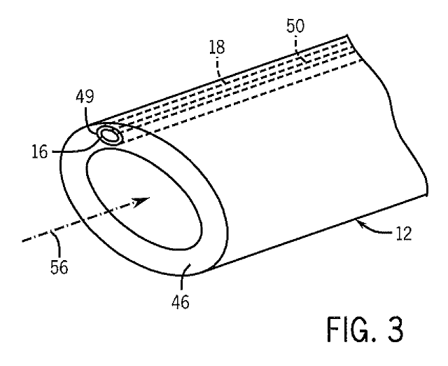

The drug delivery lumen 18 may be formed within, e.g., co-extruded with, the walls 46 of the tube 12, as shown in FIG. 3, a detail view of the distal end 20 of the tube 12 along the airflow axis 56 of the tube 12. The opening 49 in the drug delivery lumen may be located at any position on the tube 12 distal to the cuff 14. As shown, the opening 49 is formed at the distal end 20 of the tube 12. In such an embodiment, the opening 49 may be formed (or reformed) after the slant tip is cut, and the nebulizer 16 may be applied directly on the slanted end within the opening 49. In an alternative embodiment, the nebulizer 16 may be threaded into the lumen 18 from the proximal end 54. The opening 49 may be formed by cutting or forming a notch through a portion of the wall 46. While the opening 49 may be any size, its diameter may be proportional to the diameter of the lumen 18. Alternatively, a lumen 18 may have an opening 49 that is slightly larger that the diameter of the lumen 18. For example, a 1 mm lumen may have an opening 49 that is 3 mm in diameter. A slightly larger opening 49 may facilitate insertion of the nebulizer 16. In other embodiments, the drug delivery lumen 18 may be a separate structure that is adhered to or otherwise associated with the tube 12 prior to insertion. In such embodiments, the opening 18 may be preformed at an appropriate location on the structure.

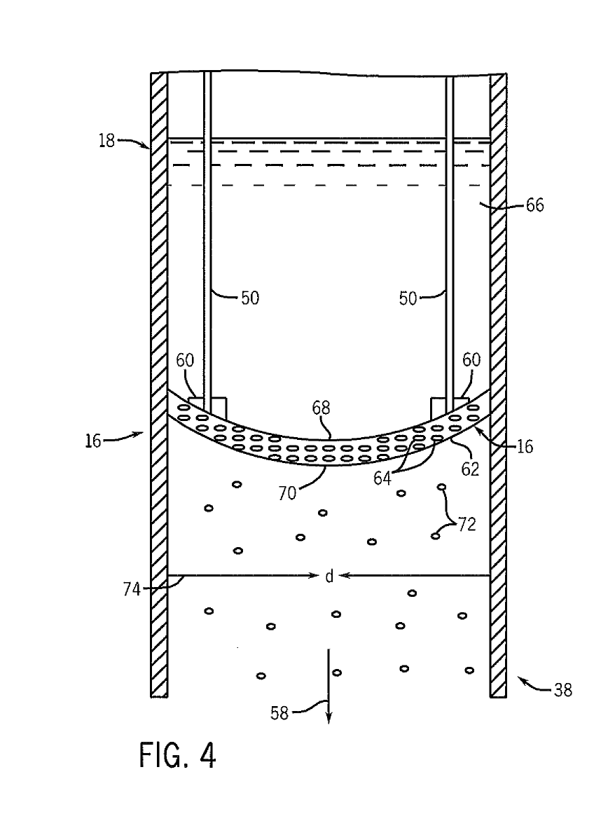

The nebulizer 16 may be any appropriate pump or other structure for dispersing a fluid as a spray having the appropriate drop size. The nebulizer 16 may include a pump (e.g., a micropump) or other micromechanical structures to facilitate forming a spray. For example, as shown in FIG. 4, a cross-sectional view of the distal end 38 of the drug delivery lumen 18, the nebulizer 16 may include a plate or membrane 62 that stretches across the diameter of the lumen 18. As shown, the membrane 62 may be generally orthogonal to the axis 58 of the lumen 18. The membrane 62 forms a seal that prevents any fluid 66 in the drug delivery lumen 18 from the traversing the membrane 62 unless the fluid 66 has passed through orifices 64.

The membrane 62 is coupled to one or more actuating devices 60. The actuating device 60 may include an electromechanical transducer unit, such as a piezoelectric element, or an ultrasonic transducer unit. When electrical current is supplied via wires 50 to the actuating device 60, the actuating device 60 causes the membrane 62 to vibrate. The fluid 66 that is on the proximal side 68 of the membrane is pushed through orifices 64 formed within the membrane 62 and emerges from the distal side 70 of the membrane 62 as a spray formed from droplets 72. The membrane includes orifices 64 of a particular size and shape to produce a desired droplet size. For example, in certain embodiments, the orifices 64 may be tapered apertures that are employed to produce small droplets. The arrangement of the nebulizer 16 may be as provided in U.S. Pat. Nos. 5,164,740, 5,586,550, and 5,758,637, the disclosures of which are hereby incorporated by reference in their entireties for all purposes.

An activation signal may be supplied to the actuating device 60 via connecting wires 50 from a controller (e.g., from monitor 30, as shown in FIG. 1). When the activation signal is supplied, the actuating device 60 oscillates, which in turn oscillates the membrane 62, and the fluid 66 is nebulized through the membrane 62. Different electrical properties of the actuating device 60 (e.g. current, voltage, phase shift) are dependant in particular on the particular features. For example, if the actuating device 60 includes an electromechanical transducer unit, the oscillation may depend on the capacitance of the structure.

In a particular embodiment, the membrane 62 may include orifices 64 that are sized and shaped to produce a plurality of droplet sizes. Particular orifices 64 may respond to different resonant frequencies. In such a manner, depending on the nature of the activation signal and/or the arrangement of the actuating devices 60, the membrane 62 may be capable of oscillation under different frequencies to produce different droplet characteristics. That is, one set of orifices 64 may respond to one frequency while another, differently-sized set of orifices 64 may respond to a second frequency. Accordingly, a single nebulizer 16 may be capable of producing multiple droplet spectrums. In other embodiments, multiple drug delivery lumens 18 may be incorporated into a single tube 12, each lumen 18 including a nebulizer 16 capable of forming droplets 72 with different sizes.

The droplet size or droplet spectrum, i.e. the distribution of the quantity of droplets of differing sizes, may be assessed by any suitable method. Various measuring methods exist for determining the droplet spectrum or the parameters describing droplet distribution, such as the mass median diameter (MMD) for example. The droplet spectrum, expressed, for example, by the MMD value, is, in one embodiment, suitable as a reference parameter for differentiating between two aerosols generated by exemplary nebulizers 16. In one embodiment, the nebulizer 16 forms droplets 72 having a MMD of equal to or less than about 5 microns. In other embodiments, the droplets 72 have a MMD of equal to or less than about 2 microns. The size of the droplets 72 may be chosen to reflect the desired tissue target. As noted, slightly larger droplets may be more appropriate for depositing in a bronchial stem, while smaller droplets may be carried into the alveoli.

The nebulizer 16 may be sufficiently small to be incorporated into a lumen 18 having a small inner diameter 74 relative to a main respiratory lumen of the tube 12. For example, the lumen 18 may have an inner diameter that is about 5 mm, 3 mm, or 1 mm or less. In this manner, the tube 12 may provide additional functionality without sacrificing a volume available for delivery respiratory gases. In particular, it is also contemplated that the tube 12 may incorporate one or more drug delivery lumens 18 in addition to any supporting lumens that may be used for suctioning secretions above the cuff 14 (see FIG. 2), inflating the cuff 14, or providing any other desired functionality. In addition, the drug delivery lumen 18 may be periodically flushed or cleaned (e.g. with saline) so that the lumen 18 may be used for the delivery of more than one drug during the course of intubation.

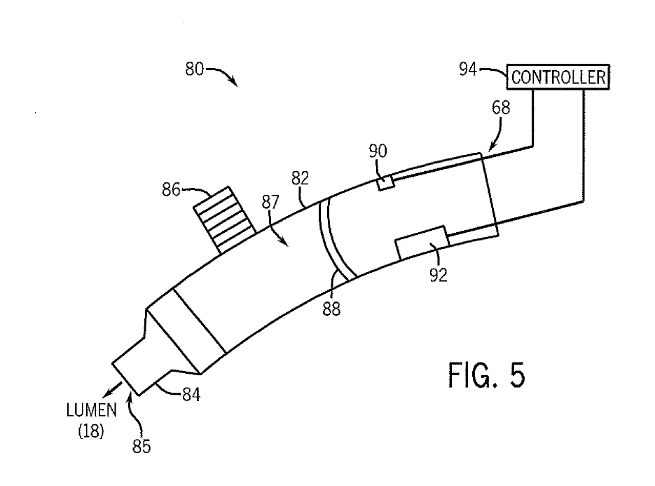

As patients are often intubated for periods of several days or weeks, it is contemplated that drug delivery may be ongoing during the intubation. As such, tracheal tubes as provided herein including a drug delivery lumen 18 and integral nebulizer 16 may be used in conjunction with suitable drug delivery monitoring devices to ensure that drug in the lumen 18 is delivered to the patient. For example, FIG. 5 illustrates a drug delivery device 80 that may be coupled to a proximal end of the drug delivery lumen 18. The drug delivery device 80 includes a conduit 82 that may be coupled to the drug delivery lumen at connector 84 located at a distal end 85 of the device 80. Drug may enter a passageway 87 through port 86, either via injection or coupling to a drug reservoir 24 (see FIG. 1). The passageway 87 is sealed at a point proximal to the port 86 via flexible seal 88.

The flexible seal 88 serves as an indirect indicator for the level of fluid in the drug delivery lumen 18. That is, when the lumen 18 is full, the flexible seal will be pushed towards the proximal end 89 of the drug delivery device 80. This in turn will result in higher pressure readings at a pressure transducer 90 located in the portion of the device proximal to the flexible seal 88. In particular embodiments, a pump 92 may apply pressure to the flexible seal 88 to push fluid through the lumen 18. Both the pressure transducer 90 and the pump 92 may be operate under a controller 94, which may be part of a standalone device or may be coupled to a patient monitor (e.g., monitor 30). In one embodiment, the pressure at the pressure transducer 90 may be used to control the pump 92. That is, when the pressure is high, the pump 92 may be activated to push the fluid through the lumen 18.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A medical device comprising:
a tracheal tube adapted to be inserted into a patient airway comprising a first lumen for delivering respiratory gases to the patient;
a second lumen formed in a wall of the tracheal tube and running substantially parallel to the first lumen, wherein the second lumen is configured to transfer a drug to a distal end of the second lumen; and
a nebulizer disposed proximate the distal end of the second lumen, wherein the nebulizer is capable of nebulizing the drug.

2. The medical device of claim 1, wherein the nebulizer comprises a membrane comprising a plurality of orifices and an actuating device configured to oscillate the membrane.

3. The medical device of claim 2, wherein when the membrane oscillates, drug that passes through the orifices from a proximal surface of the membrane to a distal surface forms a spray comprising drops.

4. The medical device of claim 3, wherein the orifices are sized and shaped so that drug that passes through the membrane forms drops having a mass median diameter of less than about 5 microns.

5. The medical device of claim 3, wherein the orifices are sized and shaped so that drug that passes through the membrane forms drops having a mass median diameter of less than about 2 microns.

6. The medical device of claim 3, wherein when the membrane oscillates at a first frequency, drug that passes through the orifices forms a spray comprising drops of a first mass median diameter and when the membrane oscillates at a second frequency, drug that passes through the orifices forms a spray comprising drops of a second mass median diameter.

7. The medical device of claim 3, comprising a controller coupled to the membrane and actuating device, wherein the controller is configured to control the actuating device.

8. The medical device of claim 1, wherein the second lumen has an inner diameter of less than about 2 millimeters.

9. The medical device of claim 1, wherein the second lumen has an inner diameter of less than about 0.75 millimeters.

10. The medical device of claim 1, wherein the drug comprises a liquid.

11. A system for drug delivery comprising:
a tracheal tube adapted to be inserted into a patient airway comprising a first lumen for delivering respiratory gases to the patient;
a second lumen formed in a wall of the tracheal tube and terminating at a distal end of the tracheal tube;
a membrane comprising a plurality of orifices disposed in a distal end of the second lumen;
an actuating device configured to oscillate the membrane; and
a controller coupled to the actuating device.

12. The system of claim 11, wherein the controller is configured to oscillate the membrane when a liquid is transferred into the second lumen.

13. The system of claim 11, wherein when the membrane oscillates, liquid that passes through the orifices from a proximal surface of the membrane to a distal surface forms a spray comprising drops.

14. The system of claim 11, wherein the orifices are sized and shaped so that liquid that passes through the membrane forms drops having a mass median diameter of less than about 5 microns.

15. The system of claim 11, wherein the orifices are sized and shaped so that liquid that passes through the membrane forms drops having a mass median diameter of less than about 2 microns.

16. The system of claim 11, wherein when the membrane oscillates at a first frequency, liquid that passes through the orifices forms a spray comprising drops of a first mass median diameter and when the membrane oscillates at a second frequency, liquid that passes through the orifices forms a spray comprising drops of a second mass median diameter.

17. A medical device comprising:
a tracheal tube adapted to be inserted into a patient airway comprising a first lumen for delivering respiratory gases to the patient;
a second lumen formed in a wall of the tracheal tube and running substantially parallel to the first lumen, wherein the second lumen is configured to transfer a liquid to a distal end of the second lumen;
a liquid entry port coupled to the second lumen;
a flexible membrane seal disposed in the second lumen at a location proximal to the liquid entry port; and
a pressure transducer coupled to the second lumen at a location proximal to the flexible membrane seal.

18. The medical device of claim 17, comprising a controller coupled to the pressure transducer, wherein the controller is configured to receive a signal from the pressure transducer related to a pressure in a space proximal to the flexible membrane seal.

19. The medical device of claim 18, wherein when the pressure in the space proximal to the flexible membrane seal is below a threshold, the controller activates a pump configured to increase the pressure against a proximal surface of the flexible membrane seal.

20. The medical device of claim 18, wherein when the pressure in the space proximal to the flexible membrane seal is below a threshold, the controller generates an indication related to the liquid.
